(19) United States
(12) Patent Application Publication
(10) Pub. No.: US 2009/0291127 A1
(43) Pub. Date: Nov. 26, 2009

(54) TRANSDERMAL ANTI-DEMENTIA ACTIVE AGENT FORMULATIONS AND METHODS FOR USING THE SAME

(76) Inventors: Jianye Wen, Palo Alto, CA (US); Yoshiko Katori, San Jose, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/437,403

(22) Filed: May 7, 2009

Related U.S. Application Data
(60) Provisional application No. 61/055,062, filed on May 21, 2008.

(51) Int. Cl. A61K 9/70; A61K 31/445; A61P 25/28

(52) U.S. Cl. 424/449; 514/319

(57) ABSTRACT

A transdermal antidementia active agent formulation is provided. In certain embodiments, the formulation includes a backing, an active agent reservoir layer including an antidementia active agent, wherein the antidementia active agent is present as both a freebase and optionally also present as a salt, an adhesive layer including the antidementia active agent, and optionally an adhesive overlay. Also provided are methods of using the formulations, e.g. for administering an antidementia active agent to a subject, and kits containing the formulations.

### CROSS-REFERENCE TO RELATED APPLICATIONS

Pursuant to 35 U.S.C. §119(e), this application claims priority to the filing date of U.S. Provisional Patent Application Ser. No. 61/055,062 filed May 21, 2008; the disclosure of which is herein incorporated by reference.

### INTRODUCTION

Alzheimer's disease is a degenerative brain disease that causes dementia, a progressive decline in cognitive function beyond what might be expected from normal aging. Short-term memory loss is the most common symptom, and later symptoms include confusion, anger, mood swings, language breakdown, long-term memory loss, and the general withdrawal of the subject as his or her senses decline. Alzheimer's disease has no current cure, however its symptoms can be treated with active agents, such as acetylcholinesterase inhibitors (e.g., donepezil, galantamine, rivastigimine, tacrine, etc.) and N-methyl D-aspartate (NMDA) receptor antagonists (e.g., memantine).

Donepezil, known chemically as (±)-2,3-dihydro-5,6-dimethoxy-2-[[1-(phenylmethyl)-4-piperidinyl]methyl]-1H-inden-1-one, is a reversible acetylcholinesterase inhibitor that is used to treat the symptoms of Alzheimer's disease. Typically, donepezil is provided as donepezil hydrochloride in tablet form for oral administration (e.g., Aricept®, Pfizer, Inc., New York).

Transdermal active agent formulations, also known as transdermal patches or skin patches, are adhesive patches containing an active agent that are placed on the skin to deliver the active agent through the skin. Transdermal patches deliver the active agent by percutaneous absorption, which is the absorption of substances through unbroken skin. After a transdermal patch is applied to the skin, the active agent contained in the patch passes through, or permeates the skin and can reach its site of action through a systemic blood flow. Alternatively, the transdermal patch may be placed on the desired treatment site such that the medication contained in the patch is delivered topically.

### SUMMARY

A transdermal antidementia active agent formulation is provided. In certain embodiments, the formulation includes a backing, an active agent reservoir layer including an antidementia active agent, wherein the antidementia active agent is present as a freebase, as a salt, or both as a freebase and salt, and an adhesive layer including the antidementia active agent. Also provided are methods of using the formulations, e.g. for administering an antidementia active agent to a subject, and kits containing the formulations.

### BRIEF DESCRIPTION OF THE FIGURES

FIG. 1 shows a cross sectional view of an embodiment of the transdermal active agent formulation described herein.

FIG. 2 shows a cross sectional view of an embodiment of the transdermal active agent formulation described herein.

FIG. 3 shows a graph of flux as a function of time for a multilaminate transdermal formulation containing an active agent reservoir layer with 6% donepezil freebase, 15% donepezil salt, and 10% SML and an adhesive layer with 6% donepezil freebase and 10% SML.

FIG. 4 shows a graph of flux as a function of time for a multilaminate transdermal formulation containing an active agent reservoir layer with 6% donepezil freebase, 15% donepezil salt, and 10% GMO and an adhesive layer with 6% donepezil freebase and 10% GMO.

FIG. 5 shows a graph of flux as a function of time for a multilaminate transdermal formulation containing an active agent reservoir layer with 6% donepezil freebase, 15% donepezil salt, and 10% LTH and an adhesive layer with 6% donepezil freebase and 10% LTH.

FIG. 6 shows a graph of flux as a function of time for a multilaminate transdermal formulation containing an active agent reservoir layer with 6% donepezil freebase, 15% donepezil salt, and 15% LTH and an adhesive layer with 6% donepezil freebase and 15% LTH.

FIG. 7 shows a graph of flux as a function of time for a single layer transdermal formulation containing 15% LTH and 6% donepezil freebase.

### DEFINITIONS

The terms “pressure-sensitive adhesive”, “self adhesive”, and “self-stick adhesive” mean an adhesive that forms a bond when pressure is applied to adhere the adhesive with a surface. Typically, no solvent, water, or heat is needed to activate the adhesive. For pressure-sensitive adhesives, the degree of bond strength is proportional to the amount of pressure that is used to apply the adhesive to the surface.

The term “saturated” means that a solution of a substance is at the saturation point, the point of maximum concentration of the substance in the solution, and the solution can not dissolve any more of that substance under normal conditions. A change in conditions my cause the concentration of the substance in the solution to be higher than the saturation point, i.e., the solution has become supersaturated.

The term “supersaturated” means that a solution contains more of the dissolved material than could be dissolved by the solvent under normal circumstances. Supersaturated solutions are prepared when some condition of a saturated solution is changed, for example temperature (e.g., by cooling), volume (e.g., by evaporation), or pressure (e.g., by compression).

### DETAILED DESCRIPTION

A transdermal antidementia active agent formulation is provided. In certain embodiments, the formulation includes a backing, an active agent reservoir layer including an antidementia active agent, wherein the antidementia active agent is present as a freebase, as a salt, or as both freebase and salt, and an adhesive layer including the antidementia active agent. Also provided are methods of using the formulations, e.g. for administering an antidementia active agent to a subject, and kits containing the formulations.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes In determining whether a number is near to or approximately a specifically recited number, the near or approximating recited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be constructed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various embodiments of the invention, aspects of the transdermal active agent formulations are reviewed first in greater detail, followed by a detailed description of methods of using the transdermal formulations and a review of kits that include the transdermal formulations.

### Transdermal Anti-Dementia Active Agent Formulations

As summarized above, transdermal antidementia active agent formulations are provided. Transdermal formulations of the invention are formulations or compositions that are configured to deliver an antidementia active agent to a subject when topically applied to a skin surface of a subject. Transdermal active agent formulations of the invention may have one or more layers (i.e., where a formulation having multiples layers is referred to herein as a formulation that includes a multilaminate design). In certain embodiments, the transdermal active agent formulations may have a backing, a polymeric active agent reservoir layer, and an adhesive layer.

In some cases, the transdermal formulations may have an intermediate layer provided between the active agent reservoir layer and the adhesive layer. In some embodiments, the intermediate layer may be a rate-controlling membrane layer. “Rate-controlling” means that the membrane meters the quantity of active agent that is administered through the skin for a prolonged period of time, such that the active agent is released from the transdermal formulation at a substantially constant rate until the desired total quantity (i.e., target dosage) of active agent is administered.

In other embodiments, the intermediate layer may be a non-rate controlling layer. “Non-rate controlling” means that the layer does not significantly affect the flux or the release of the active agent from the transdermal formulation.

In some embodiments, a release liner is provided on the adhesive layer, specifically on a surface of the adhesive layer that is distal from the reservoir layer. The release liner facilitates the protection of the active agent reservoir layer and the adhesive layer. Prior to application onto a skin surface, the release liner may be removed, thereby exposing the adhesive layer. The release liner may be prepared by treating one side of polyethylene-coated wood free paper, polyolefin-coated glassine paper, a polyethylene terephthalate (polyester) film, a polypropylene film, or the like with a silicone treatment.

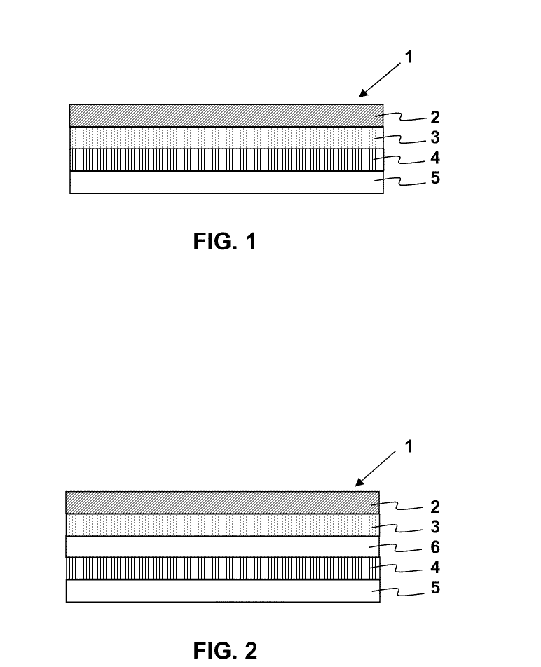

FIG. 1 shows an embodiment of the transdermal active agent formulation 1, where the transdermal active agent formulation 1 includes a backing layer 2, an active agent reservoir layer 3, an adhesive layer 4, and a release liner 5. FIG. 2 shows an alternative embodiment of the transdermal active agent formulation 1, where the transdermal active agent formulation I includes a backing layer 2, an active agent reservoir layer 3, an intermediate layer 6, an adhesive layer 4, and a release liner 5. In these embodiments, the intermediate layer may be a rate-controlling membrane or a non-rate controlling layer.

The active agent reservoir layer and the adhesive layer may contain an antidementia active agent, such as donepezil, galantamine, rivastigimine, tacrine, memantine, or the like. Because the active agent is applied topically, the active agent may be present as a freebase to facilitate permeation of the active agent through the skin. In some embodiments, the antidementia active agent is present as both a freebase and a salt. In these embodiments, the antidementia active agent may be donepezil freebase and donepezil hydrochloride. Donepezil freebase has the empirical formula of C₂₄H₂₉NO₃ and the IUPAC name (±)-2,3-dihydro-5,6-dimethoxy-2-[[1-(phenylmethyl)-4-piperidinyl]methyl]-1H-inden-1-one. Donepezil has the following chemical structure:

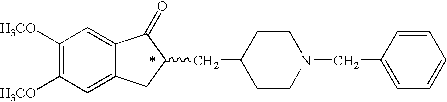

Salts of donepezil may include the hydrochloride salt, and the like. Donepezil hydrochloride salt, or donepezil-HCl, has the empirical formula of C₂₄H₂₉NO₃.HCl and the IUPAC name (±)-2,3-dihydro-5,6-dimethoxy-2-[[1-(phenylmethyl)-4-piperidinyl]methyl]-1H-inden-1-one hydrochloride. Donepezil-HCl has the following chemical structure:

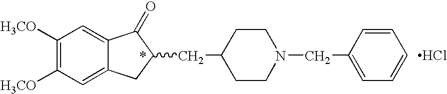

As indicated above, the antidementia active agent is present as both a freebase and a salt, such as but not limited to donepezil freebase and donepezil-HCl. An aspect of the subject formulations is that the active agent reservoir layer may be saturated or supersaturated with donepezil freebase or both donepezil freebase and donepezil salt. In certain embodiments, the adhesive layer may be saturated or supersaturated with donepezil freebase freebase or both donepezil freebase and donepezil salt.

In certain embodiments, the transdermal active agent formulation may be provided in the form of an adhesive tape or an adhesive patch. In these embodiments, the transdermal active agent formulation may be applied to a skin surface such that the adhesive layer is adhered to a skin surface by the adhesion of the adhesive to the skin surface. In certain cases, the transdermal active agent formulation is an adhesive patch preparation that includes a pressure-sensitive adhesive. In these cases, the transdermal active agent formulation may be prepared in accordance with the solvent coating method, or the like.

The transdermal active agent formulations may be adhered to the skin for periods of time, for instance 3 days or greater, including 7 days or greater, such as 10 days or greater. A feature of the subject formulations is that they are configured to provide for a skin permeation rate (i.e., transdermal flux rate) sufficient to administer a target dosage of active agent to a subject over a period of time. In some cases, the target dosage of the active agent may be 5 mg/day or greater over a one week period (i.e., 7 days or 168 hours), including 10 mg/day or greater over one week, such as 15 mg/day or greater over one week. In some cases the maximal skin permeation rate of the active agent may be about 2.8 μg/cm²/hr or greater, including about 4.8 μg/cm²/hr or greater, or about 6.2 μg/cm²/hr or greater, such as about 6.7 μg/cm²/hr or greater. Transdermal flux rates may be determined using the procedure described in examples.

The size (i.e., area) of the transdermal patch depends on the transdermal flux rate of the active agent and the target dosage. For example, if the transdermal flux is 4.8 μg/cm²/hr and the target dosage is 5 mg/day, then the transdermal patch would have an area of about 43 cm². Or for example, if the transdermal flux is 4.8 μg/cm²/hr and the target dosage is 10 mg/day, then the transdermal patch would have an area of about 87 cm². In addition, the total drug loading in a patch is dependent on patch thickness. Excess drug loading is employed in certain embodiments to maintain a steady state flux and deliver a targeted amount of drug in the desired delivery period. Exemplary multilaminate formulations with donepezil freebase and donepezil-HCl are shown in Table 1 below.

TABLE 1
Multilaminate Formulations with Donepezil Freebase and Donepezil-HCl

| Target | | | | | | |
|---|---|---|---|---|---|---|
| Dosage | Flux | Patch Size | Drug Loading | Drug | Drug Loading | Thickness |
| (mg/day) | (μg/cm²/hr) | (cm²) | (wt %) | utilization | for 7 days (mg) | (μm) |
| 10 | 2.8 | 149 | 14 | 0.3 | 233 | 112 |
| 5 | 4.8 | 43 | 14 | 0.3 | 117 | 192 |
| 10 | 4.8 | 87 | 14 | 0.3 | 233 | 192 |
| 10 | 6.7 | 62 | 14 | 0.3 | 233 | 268 |

### Backing Layer

The transdermal active agent formulation that is employed herein may have a backing layer. The backing may be flexible to an extent that it can be brought into close contact with a skin surface. The backing is such that it does not absorb the active agent, and does not allow the active agent to be released from the backing side. The backing may include, but is not limited to, non-woven fabrics, fabrics, films (including sheets), porous bodies, foamed bodies, paper, composite materials obtained by laminating a film on a non-woven fabric or fabric, and combinations thereof.

Non-woven fabric may include, but is not limited to the following: polyolefin resins such as polyethylene and polypropylene; polyester resins such as polyethylene terephthalate, polybutylene terephthalate and polyethylene naphthalate; and besides rayon, polyamide, poly(ester ether), polyurethane, polyacrylic resins, polyvinyl alcohol, styrene-isoprene-styrene copolymers, and styrene-ethylene-propylene-styrene copolymers; and combinations thereof. Fabric may include, but is not limited to cotton, rayon, polyacrylic resins, polyester resins, polyvinyl alcohol, and combinations thereof.

The film may include, but is not limited to the following: polyolefin resins such as polyethylene and polypropylene; polyacrylic resins such as polymethyl methacrylate and polyethyl methacrylate; polyester resins such as polyethylene terephthalate, polybutylene terephthalate and polyethylene naphthalate; and besides cellophane, polyvinyl alcohol, ethylene-vinyl alcohol copolymers, polyvinyl chloride, polystyrene, polyurethane, polyacrylonitrile, fluororesins, styrene-isoprene-styrene copolymers, styrene-butadiene rubber, polybutadiene, ethylene-vinyl acetate copolymers, polyamide, and polysulfone; and combinations thereof.

The paper may include, but is not limited to impregnated paper, coated paper, wood free paper, Kraft paper, Japanese paper, glassine paper, synthetic paper, and combinations thereof Composite materials may include, but are not limited to composite materials obtained by laminating the above-described film on the above-described non-woven fabric or fabric.

### Active Agent Reservoir Layer

The transdermal active agent formulation that is employed herein may have an active agent reservoir layer provided on the backing. The active agent reservoir layer may contain donepezil freebase and donepezil hydrochloride. In certain cases, the active agent reservoir may be saturated or supersaturated with donepezil freebase or donepezil freebase and donepezil salt.

Polymeric materials are used as the carrier for carrying active agents or its salts or/and optionally other ingredients in the reservoir. These materials include but not limited to polyacrylates, ethylene vinyl acetate (EVA) copolymer, and polyurethanes. EVA copolymers are thermoplastic hot-melt adhesives. EVA copolymers are conventionally considered to be copolymers of ethylene and vinyl acetate. Generally, the vinyl acetate content is about 4 wt % to 50 wt %, such as about 10 wt % to 49 wt %. Ethylene-vinyl acetate copolymers EVA materials are commercially available from various suppliers, e.g., Minnesota Mining Co.

In some embodiments, the active agent reservoir layer may contain an aminated polymer, such as but not limited to a copolymer of methyl methacrylate, butyl methacrylate, and dimethylaminoethyl methacrylate (commercially available as Eudragit® E100, Evonik Industries AG, Essen, Germany). In these cases, the aminated polymer may be basic, such that the aminated polymer facilitates the conversion of donepezil hydrochloride to donepezil freebase. Consequently, the active agent reservoir layer may remain saturated or supersaturated with donepezil freebase as donepezil freebase is absorbed from the transdermal patch through the skin of the subject.

The transdermal active agent formulation as described herein may contain a percutaneous absorption enhancer. The percutaneous absorption enhancer may facilitate the absorption of the active agent by the skin of the subject. The percutaneous absorption enhancer may also be referred to as a percutaneous permeation enhancer because it may facilitate not only the percutaneous absorption of the active agent, but also the percutaneous permeation of the active agent through the skin of the subject.

In some cases, the percutaneous absorption enhancer may be provided in at least one of the active agent reservoir layer and the adhesive layer. For example, the active agent reservoir layer may contain the percutaneous absorption enhancer, the adhesive layer may contain the percutaneous absorption enhancer, or both the active agent reservoir layer and the adhesive layer may contain the percutaneous absorption enhancer.

The percutaneous absorption enhancer may include, but is not limited to the following: aliphatic alcohols, such as but not limited to saturated or unsaturated higher alcohols having 12 to 22 carbon atoms, such as oleyl alcohol and lauryl alcohol; fatty acids, such as but not limited to linolic acid, oleic acid, linolenic acid, stearic acid, isostearic acid and palmitic acid; fatty acid esters, such as but not limited to isopropyl myristate, diisopropyl adipate, and isopropyl palmitate; alcohol amines, such as but not limited to triethanolamine, triethanolamine hydrochloride, and diisopropanolamine; polyhydric alcohol alkyl ethers, such as but not limited to alkyl ethers of polyhydric alcohols such as glycerol, ethylene glycol, propylene glycol, 1,3-butylene glycol, diglycerol, polyglycerol, diethylene glycol, polyethylene glycol, dipropylene glycol, polypropylene glycol, sorbitan, sorbitol, isosorbide, methyl glucoside, oligosaccharides, and reducing oligosaccharides, where the number of carbon atoms of the alkyl group moiety in the polyhydric alcohol alkyl ethers is preferably 6 to 20; polyoxyethylene alkyl ethers, such as but not limited to polyoxyethylene alkyl ethers in which the number of carbon atoms of the alkyl group moiety is 6 to 20, and the number of repeating units (e.g. —O—CH₂CH₂—) of the polyoxyethylene chain is 1 to 9, such as but not limited to polyoxyethylene lauryl ether, polyoxyethylene cetyl ether, polyoxyethylene stearyl ether, and polyoxyethylene oleyl ether; glycerides (i.e., fatty acid esters of glycerol), such as but not limited to glycerol esters of fatty acids having 6 to 18 carbon atoms, where the glycerides may be monoglycerides (i.e., a glycerol molecule covalently bonded to one fatty acid chain through an ester linkage), diglycerides (i.e., a glycerol molecule covalently bonded to two fatty acid chains through ester linkages), triglycerides (i.e., a glycerol molecule covalently bonded to three fatty acid chains through ester linkages), or combinations thereof, where the fatty acid components forming the glycerides include, but are not limited to octanoic acid, decanoic acid, dodecanoic acid, tetradecanoic acid, hexadecanoic acid, octadecanoic acid (i.e., stearic acid) and oleic acid; middle-chain fatty acid esters of polyhydric alcohols; lactic acid alkyl esters; dibasic acid alkyl esters; acylated amino acids; pyrrolidone; pyrrolidone derivatives; and combinations thereof.

Additional types of percutaneous absorption enhancers include, but are not limited to lactic acid, tartaric acid, 1,2,6-hexanetriol, benzyl alcohol, lanoline, potassium hydroxide (KOH), and tris(hydroxymethyl)aminomethane.

Specific examples of percutaneous absorption enhancers include, but are not limited to glycerol monooleate (GMO), sorbitan monolaurate (SML), sorbitan monooleate (SMO), laureth-4 (LTH), and combinations thereof.

In some cases, the active agent reservoir layer contains the percutaneous absorption enhancer in an amount ranging from 2% to 25% (w/w), such as from 5% to 20% (w/w), and including from 5% to 15% (w/w). In certain cases, the active agent reservoir layer contains the percutaneous absorption enhancer in an amount of about 5% (w/w), about 10% (wlw), about 15% (wlw), or about 20% (w/w).

### Adhesive Layer

The transdermal active agent formulation that is employed herein may have an adhesive layer for facilitating adhesion of the transdermal patch to the skin of the subject. In certain embodiments, the adhesive layer may be provided on the active agent reservoir layer. In other cases, an intermediate layer such as a rate-controlling membrane or a non-rate controlling layer may be provided between the active agent reservoir layer and the adhesive layer. The adhesive layer itself may also serve as rate controlling layer in certain embodiments. The adhesive layer may contain donepezil freebase, as described in more detail below. In certain cases, the adhesive layer may be saturated or supersaturated with donepezil freebase. In some cases, the adhesive layer may be an acrylic pressure-sensitive adhesive layer that contains donepezil freebase. In certain embodiments, the acrylic pressure-sensitive adhesive is a copolymer of an acrylate and at least one other monomer, e.g. vinyl acetate, butyl acrylate, 2-ethylhexyl acrylate, hydroxyethyl acrylate, t-octyl acrylamide, methyl methacrylate, and acrylic acid or (meth)acrylic acid. In certain cases, the acrylic pressure-sensitive adhesive may be an acrylate-vinyl acetate copolymer, in an organic solvent solution. In these embodiments, the organic solvent may include, but is not limited to ethyl acetate, isopropyl alcohol, hexane, heptane, toluene, and combinations thereof. In some cases, the organic solvent may be ethyl acetate.

In some embodiments, the adhesive may have a composition that is substantially the same as the composition of DuroTak® 87-2287 (National Adhesives, Bridgewater, N.J.). The term “substantially the same” as used herein refers to a composition that is an acrylate-vinyl acetate copolymer in an organic solvent solution and provides for the functionality as described herein In some embodiments, the acrylic pressure-sensitive adhesive is DuroTak® 87-2287.

In some embodiments, the adhesive may have a composition that is substantially the same as the composition of or DuroTak® 87-4287 (National Adhesives, Bridgewater, N.J.). The term “substantially the same” as used herein refers to a composition that is an acrylate-vinyl acetate copolymer in an organic solvent solution and provides for the functionality as described herein. In some embodiments, the acrylic pressure-sensitive adhesive is or DuroTak® 87-4287. Other examples of polyacrylate-based adhesives are as follows, identified as product numbers, manufactured by National Starch (DURO-TAK® is a trademark of National Starch adhesives): 87-4098, 87-2516, 87-2051, 87-2052, 87-2054, 87-2196, 87-9259, 87-9261, 87-2979, 87-2510, 87-2353, 87-2100, 87-2852, 87-2074, 87-2258, 87-9085, 87-9301 and 87-5298. DURO-TAK® 87-2287 and 87-4287 both are polymeric adhesives derived from monomer compositions that are similar.

In some cases, the adhesive layer may further include a percutaneous absorption enhancer, as described above. The percutaneous absorption enhancer in the adhesive layer may be the same or different from the percutaneous absorption enhancer in the active agent reservoir layer. In some cases, the adhesive layer contains the percutaneous absorption enhancer in an amount ranging from 2% to 25% (w/w), such as from 5% to 20% (w/w), including from 5% to 15% (w/w). In certain cases, the adhesive layer contains the percutaneous absorption enhancer in an amount of about 5% (w/w), about 10% (w/w), about 15% (w/w), or about 20% (w/w).

In one type of embodiments, the adhesive composition of this invention may contain polyisobutylene (PIB). PIB is typically a blend of high molecular weight PIB and low molecular weight PIB. As an example, in one effective embodiment the PIB adhesive includes 8 wt % high molecular weight (such as OPPANOL L80, L100, and L140 from BASF) PIB material and 92 wt % low molecular weight (Such as OPPANOL B10, B11, B12, and B13 from BASF) PIB material. The PIB can be with or without tackifiers or plasticizers, such as low molecular weight polybutene (e.g., INDOPOL H1900 and/or high Tg, low molecular weight aliphatic resins such as the ESCOREZ resins available from Exxon Chemical, and the like).

Another kind of adhesive that can be used is a silicone adhesive. The silicone adhesives that may be used are typically high molecular weight polydimethyl siloxanes or polydimethyidiphenyl siloxanes. Formulations of silicone adhesives that are useful in transdermal patches are described in U.S. Pat. Nos. 5,232,702, 4,906,169 and 4,951,622. One example of such a silicone adhesive is Silicone 4202 polydimethylsiloxane adhesive from Dow Corning.

### Rate-Controlling Membrane

The transdermal active agent formulation that is employed herein may have an intermediate layer provided between the active agent reservoir layer and the adhesive layer. In some embodiments, the intermediate layer may be a rate-controlling membrane. The rate-controlling membrane meters the quantity of active agent that is administered through the skin for a prolonged period of time, such that the active agent is released from the transdermal formulation at a substantially constant rate until the desired total quantity (i.e., target dosage) of active agent is administered.

In certain embodiments, the rate-controlling membrane may be a microporous membrane having pores that allow permeation of the active agent. In these embodiments, the flux or release rate of the active agent by the membrane is controlled by the rate of which the active agent is able to diffuse through the pores of the membrane. The rate-controlling membrane may be any porous material that permits the permeation of the active agent, such as but not limited to polypropylene, polyethylene, polyacrylonitrile, polytetrafluoroethylene, polydimethylsiloxane, polymethyl methacrylate, and combinations thereof. Additionally, the rate-controlling membrane may be single layer or multi-layer (i.e., having one or more microporous membrane layers composed of the same or different material laminated together). In certain embodiments, the rate-controlling membrane is a monolayer polypropylene membrane.

The porosity, pore size and thickness of the rate-controlling membrane depend on the physicochemical properties, such as the molecular weight of the active agent, the flux required, and the like. For example, the rate-controlling membrane may typically have the following properties: a porosity ranging from about 10% to 85%, including from about 20% to 75%, such as from 30% to 50%; a pore size ranging from 0.03-0.25 μm×μm, including 0.03-0.2 μm×μm, such as 0.04-0.12 μm×μm; and a thickness ranging from 10 μm to 70 μm, including from 15 μm to 60 μm, such as from 20 μm to 50 μm. In certain embodiments, the rate-controlling membrane may have a porosity of 37%, a pore size of 0.04-0.12 μm×μm, and a thickness of 25 μm.

In some embodiments, the rate-controlling membrane may have a composition that is substantially the same as the composition of Celgard® 2400 (Celgard LLC, Charlotte, N.C.). The term “substantially the same” as used herein refers to a composition that is a monolayer polypropylene membrane and provides for the functionality as described herein. In some embodiments, the rate-controlling membrane is Celgard® 2400.

Non-Rate Controlling Layer The transdermal active agent formulation that is employed herein may have an intermediate layer provided between the active agent reservoir layer and the adhesive layer. In some embodiments, the intermediate layer may be a non-rate controlling layer. The non-rate controlling layer does not significantly affect the flux or the release of the active agent from the transdermal formulation. In certain embodiments, the non-rate controlling layer may facilitate the reduction of cold flow (i.e., the movement of material over a period of time) of the layers of the transdermal formulation. In these embodiments, the non-rate controlling layer may be a non-woven layer, such as but not limited to non-woven polyester fabric from Reeway inc., and combinations thereof.

### Adhesive Overlay

Optionally, the overlay can be used to increase the adhesion of the kits. Overlay can be but not limited to a layer of adhesive on porous, non-porous, occlusive, or breathable backing materials. The overlay can be applied by the patients or can be integrated in the kits.

### Anti-Dementia Active Agent

As reviewed above, the antidementia active agent of the subject formulations can be donepezil. Donepezil may be present as both a freebase and a salt, such as but not limited to donepezil freebase and donepezil hydrochloride. In certain embodiments, the active agent reservoir layer is saturated or supersaturated with donepezil freebase or donepezil freebase and donepezil salt. In some cases, the active agent reservoir layer contains donepezil freebase in an amount ranging from 1% to 25% (w/w), such as from 2% to 20% (w/w), including from 5% to 20% (w/w), and donepezil hydrochloride in an amount ranging from 2% to 30% (w/w), such as from 5% to 25% (w/w), including from 5% to 20% (w/w). In certain cases, the active agent reservoir layer contains donepezil freebase in an amount of 6% (w/w) and donepezil hydrochloride in an amount of 15% (w/w).

As indicated above, in certain embodiments, the adhesive layer may contain donepezil freebase. In these embodiments, the adhesive layer may be saturated or supersaturated with donepezil freebase. In some cases, the adhesive layer contains donepezil freebase in an amount ranging from 1% to 25% (w/w), such as from 2% to 10% (w/w), including from 5% to 7% (w/w). In certain cases, the active agent reservoir layer contains donepezil freebase in an amount of 6% (w/w).

### Methods

Methods for administering an antidementia agent to a subject are also provided. In certain embodiments, the method includes applying to a skin site of the subject a transdermal antidementia active agent formulation as described in detail above, and maintaining the formulation at the skin site of the subject for a period of time sufficient to deliver the active agent to the subject. The transdermal active agent formulation may be applied to the skin of the subject, for example at a skin site, a keratinized skin site, etc. The transdermal active agent formulation may be applied to a skin surface such that the formulation is adhered to a skin surface by the adhesion of the adhesive layer to the skin surface.

In some cases, the transdermal active agent formulation may be applied to a skin site for an amount of time sufficient to deliver the active agent to the subject. In some cases, the transdermal active agent formulation may be applied to the skin site for an amount of time sufficient to deliver an effective amount of the active agent to the subject. The term “effective amount” means a dosage sufficient to provide the desired result. For example, an effective amount may be an amount of the active agent present in the formulation that is sufficient such that, when applied to a skin site in accordance with the methods described herein, the subject's symptoms associated with Alzheimer's disease and/or dementia are treated.

In some embodiments, the transdermal active agent formulation may be applied to the skin site for an amount of time sufficient to deliver a target dose of the active agent to the subject over a period of time. For example, the target dose of the active agent may be 5 mg/day or greater, including 10 mg/day or greater, such as 15 mg/day or greater. In some cases, the transdermal active agent formulation may be applied to the skin site for an amount of time ranging from 1 day to 14 days, such as 3 days to 10 days, including 7 days to 10 days. In certain cases, the transdermal active agent formulation may be applied to the skin site for 7 days (i.e., one week).

After the transdermal active agent formulation has been applied to the skin site for the desired amount of time (i.e., an amount of time sufficient to deliver a target dose of the active agent to the subject over a period of time), the formulation may be removed from the skin site. A new transdermal formulation may be applied at the same or at a different skin site. The new transdermal formulation may be applied to a different skin site to reduce the possible occurrence of skin irritation and/or skin sensitization at the prior site of application.

In certain embodiments, the methods described herein may include a diagnostic step. Individuals may be diagnosed as being in need of the subject methods using any convenient protocol, and are generally known to be in need of the subject methods, e.g., they are suffering from a target disease condition or have been determined to be at risk for suffering from a target disease condition, prior to practicing the subject methods.

Diagnosis or assessment of Alzheimer's disease and dementia is well-established in the art. Assessment may be performed based on, but not limited to the following: patient history; collateral history from relatives; diagnostic tests, such as clinical observation of behavior; mental status testing of cognitive functions including but not limited to memory, language, perceptual skills, attention, constructive abilities, orientation, problem solving and functional abilities; physical examinations; neurological examinations; brain imaging, such as but not limited to computed tomography (CT), magnetic resonance imaging (MRI), positron emission tomography (PET), and single photon emission computed tomography (SPECT); and the like.

### Utility

The transdermal active agent formulations find use in any application where a subject would benefit from being administered an antidementia active agent, such as but not limited to donepezil. In certain embodiments, the formulations are employed in the treatment of a condition. By treatment is meant that at least an amelioration of the symptoms associated with the condition afflicting the subject is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g., terminated, such that the subject no longer suffers from the condition, or at least the symptoms that characterize the condition.

In general, administration of donepezil according to the subject methods can be used to treat diseases or conditions including, but not limited to Alzheimer's disease, dementia, and the like. The transdermal active agent formulation may be used for administering donepezil to a subject. In these cases, the method includes applying a transdermal active agent formulation, as described herein, to a skin surface of a subject. The method further includes maintaining the active agent formulation on the skin of the subject for a period of time sufficient to deliver the active agent to the subject. Subjects may include humans or animals, such as but not limited to mice, rats, dogs, rabbits, and the like.

In certain embodiments, the transdermal active agent formulation is provided as an adhesive patch and is applied to the skin surface, whereby the active agent in the formulation can be administered by percutaneous permeation through the skin. When the transdermal active agent formulation is applied to a skin surface, the active agent permeates the skin in contact with the patch to reach the site of action through a systemic blood flow.

### Kits

Kits for use in practicing the methods described herein are also provided. In certain embodiments, the kits include a transdermal active agent formulation that includes a backing layer, a polymeric active agent reservoir layer provided on the backing layer, and an adhesive layer. In these embodiments, the active agent reservoir layer includes an antidementia active agent present as both a freebase and a salt, as described above. Additionally, the adhesive layer contains donepezil freebase, as described above.

In certain embodiments, the kits provide for maximal skin permeation rates of the antidementia active agent after applying to the skin of about 2.8 μg/cm²/hr or greater, including about 4.8 μg/cm²/hr or greater, or about 6.2 μg/cm²/hr or greater, such as about 6.7 μg/cm²/hr or greater.

In certain embodiments, the kits will further include instructions for practicing the subject methods or means for obtaining the same (e.g., a website URL directing the user to a webpage which provides the instructions), where these instructions may be printed on a substrate, where substrate may be one or more of: a package insert, the packaging, reagent containers and the like. In the subject kits, the one or more components are present in the same or different containers, as may be convenient or desirable.

The following examples are offered by way of illustration and not by way of limitation. Specifically, the following examples are of specific embodiments for carrying out the present invention. The examples are for illustrative purposes only, and are not intended to limit the scope of the present invention in any way.

### EXAMPLES

### I. Materials and Methods

### A. Preparation of Active Agent Reservoir Layer

Formulations were prepared by mixing stock solutions of each of the mixture components in organic solvents (typically 50-60 wt % solid content in ethyl acetate, methanol and/or ethanol), followed by a mixing process. Once a homogeneous mixture was formed, the solution was cast on a release liner (sliconized polyester sheet of 2-3 mils) and dried at 65° C. for 90 minutes. The adhesive films were laminated to a PET backing.

### B. Preparation of Adhesive Layer and a Transdermal Anti-Dementia Active Agent Formulation Preparation

Formulations were prepared by mixing stock solutions of each of the mixture components in organic solvents (typically 50-60 wt % solid content in ethyl acetate, methanol and/or ethanol), followed by a mixing process. Once a homogeneous mixture was formed, the solution was cast on a release liner (sliconized polyester sheet of 2-3 mils) and dried at 65° C. for 90 minutes. The adhesive films were laminated to another release liner.

When needed, the active agent reservoir layer can be laminated with adhesive layer with or without the presence of a rate-control or non-rate -control membrane by removing the release liner from both layer and put them together.

### C. Transdermal Flux Tests

Human cadaver skin was used and epidermal layers (stratum corneum and viable epidermis) were separated from the full-thickness skin as skin membrane. Samples were die-cut with an arch punch to a final diameter of about 2.0 cm². The release liner was removed and the system was placed on top of the epidermis/stratum corneum with the drug adhesive layer facing the stratum corneum. Gentle pressure was applied to effect good contact between the adhesive layer and stratum corneum. The donor and receptor sides of the Franz cell were clamped together and the receptor solution containing a phosphate buffer at pH 6.5 was added to the Franz cell. The cells were kept at 35° C. for the duration of the experiment. Samples of the receptor solution were taken at regular intervals and the active agent concentration was measured by HPLC. The removed receptor solution was replaced with fresh solution to maintain the sink conditions. The flux was calculated from the cumulative amounts of the drug in the receiver compartment versus time.

### II. Specific Examples

### Example 1

### Flux of Transdermal System Containing Sorbitan Monolaurate (SML)

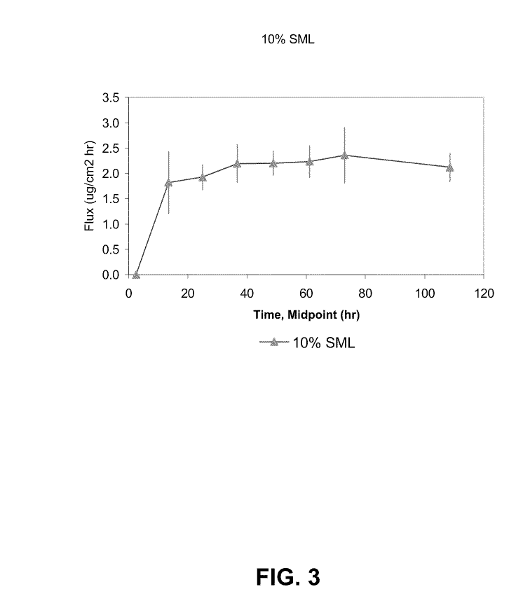

Using the general method described previously, a transdermal system containing 10% SML were prepared with details shown in following table. The steady state flux through human cadaver skin was estimated from FIG. 3 to be around 2.3 μg/cm².hr.

TABLE

| | | | Steady |
|---|---|---|---|
| | Formulation | | state flux, |
| Sample | Adhesive layer | Drug layer | μg/cm²hr |
| 10% SML | Duro-tak 87- | Duro-tak 87-2516 10% SML | 2.3 |
| | 4287 10% SML | 15% Donepezil HCl, 6% | |
| | 6% Donepezil | Donepezil base, 10.7% | |
| | base | Eudragit E100 | |

### Example 2

### Flux of Transdermal System Containing Glycerol Monooleate (GMO)

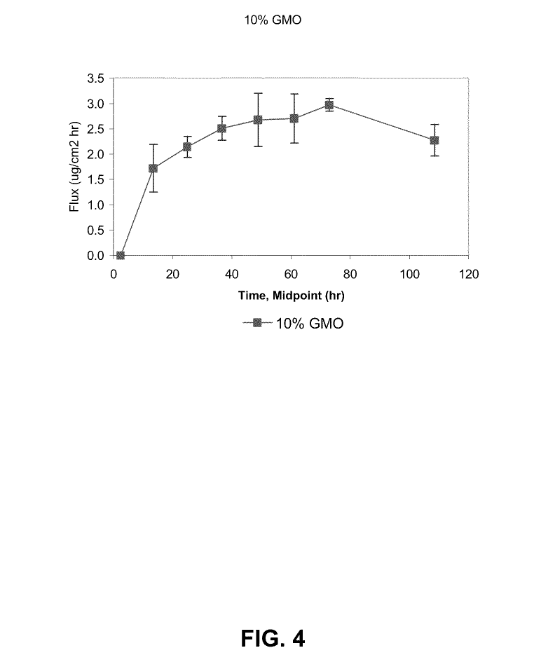

Using the general method described previously, a transdermal system containing 10% GMO were prepared with details shown in following table. The steady state flux through human cadaver skin was estimated from FIG. 4 to be around 2.7 μg/cm².hr.

TABLE

| | | | Steady |
|---|---|---|---|
| | Formulation | | state flux, |
| Sample | Adhesive layer | Drug layer | μg/cm²hr |
| 10% | Duro-tak 87- | Duro-tak 87-2516 10% GMO | 2.7 |
| GMO | 4287 10% GMO | 15% Donepezil HCl, 6% | |
| | 6% Donepezil | Donepezil base, 10.7% | |
| | base | Eudragit E100 | |

### Example 3

### Flux of Transdermal System Containing Laureth-4 (LTH)

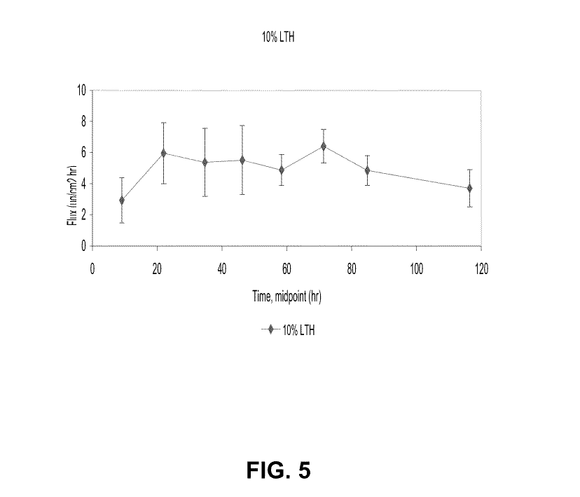

Using the general method described previously, a transdermal system containing 10% LTH were prepared with details shown in following table. The steady state flux through human cadaver skin was estimated from FIG. 5 to be around 5.5 μg/cm².hr.

TABLE

| | | | Steady |
|---|---|---|---|
| | Formulation | | state flux, |
| Sample | Adhesive layer | Drug layer | μg/cm²hr |
| 10% LTH | Duro-tak 87- | Duro-tak 87-2516 10% | 5.5 |
| | 4287 10% LTH | LTH 15% Donepezil HCl, 6% | |
| | 6% Donepezil | Donepezil base, 10.7% | |
| | base | Eudragit E100 | |

### Example 4

### Flux of Transdermal System Containing Laureth-4 (LTH)

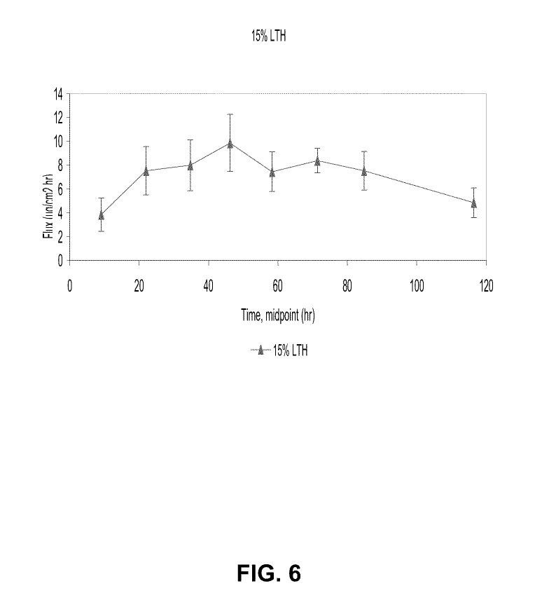

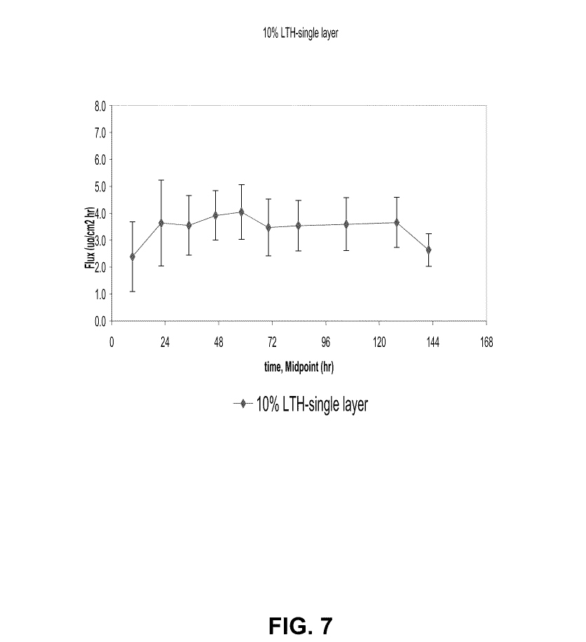

Using the general method described previously, a transdermal system containing 15% LTH were prepared with details shown in following table. The steady state flux through human cadaver skin was estimated from FIG. 6 to be around 8.4 μg/cm².hr.

TABLE

| | | | Steady |
|---|---|---|---|
| | Formulation | | state flux, |
| Sample | Adhesive layer | Drug layer | μg/cm²hr |
| 10% LTH | Duro-tak 87- | Duro-tak 87-2516 15% LTH | 8.4 |
| | 4287 15% LTH | 15% Donepezil HCl, 6% | |
| | 6% Donepezil | Donepezil base, 10.7% | |
| | base | Eudragit E100 | |

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A transdermal antidementia active agent formulation, said formulation comprising:
a backing;
an active agent reservoir layer comprising an antidementia active agent, wherein said antidementia active agent is present as a freebase; and
an adhesive layer comprising said antidementia active agent.

2. The formulation according to claim 1, wherein said antidementia active agent is donepezil.

3. The formulation according to claim 2, wherein said active agent reservoir layer is saturated with donepezil freebase or donepezil freebase and donepezil salt.

4. The formulation according to claim 2, wherein said active agent reservoir layer comprises donepezil freebase in an amount ranging from 1% to 25% (w/w) and donepezil hydrochloride in an amount ranging from 5% to 25% (w/w).

5. The formulation according to claim 4, wherein said active agent reservoir layer comprises donepezil freebase in an amount of 6% (w/w) and donepezil hydrochloride in an amount of 15% (w/w).

6. The formulation according to claim 1, wherein said antidementia active agent in said adhesive layer is present as a freebase.

7. The formulation according to claim 6, wherein said antidementia active agent in said adhesive layer is donepezil.

8. The formulation according to claim 7, wherein said adhesive layer comprises donepezil freebase in an amount ranging from 1% to 25% (w/w).

9. The formulation according to claim 8, wherein said adhesive layer comprises donepezil freebase in an amount of 6% (w/w).

10. The formulation according to claim 1, wherein at least one of said active agent reservoir layer and said adhesive layer further comprise a percutaneous absorption enhancer.

11. The formulation according to claim 10, wherein said percutaneous absorption enhancer is glycerol monooleate, sorbitan monolaurate, sorbitan monooleate, laureth-4, or a combination thereof.

12. The formulation according to claim 10, wherein said active agent reservoir layer comprises said percutaneous absorption enhancer.

13. The formulation according to claim 10, wherein said adhesive layer comprises said percutaneous absorption enhancer.

14. The formulation according to claim 10, wherein said active agent reservoir layer and said adhesive layer comprise said percutaneous absorption enhancer.

15. The formulation according to claim 1, wherein said active agent reservoir layer comprises an aminated polymer.

16. The formulation according to claim 15, wherein said aminated polymer is a copolymer comprising methyl methacrylate, butyl methacrylate, and dimethylaminoethyl methacrylate.

17. The formulation according to claim 1, wherein said adhesive layer comprises an acrylate-vinylacetate copolymer adhesive.

18. The formulation according to claim 1, further comprising a rate-controlling membrane provided between said active agent reservoir layer and said adhesive layer.

19. The formulation according to claim 1, further comprising a non-rate controlling layer provided between said active agent reservoir layer and said adhesive layer.

20. The formulation according to claim 1, wherein said formulation is configured to provide a maximal skin permeation rate of said antidementia active agent of 2.8 μg/cm²/hr or greater.

21. The formulation according to claim 20, wherein said formulation is configured to provide a maximal skin permeation rate of said antidementia active agent of 4.8 μg/cm²/hr or greater.

22. The formulation according to claim 21, wherein said formulation is configured to provide a maximal skin permeation rate of said antidementia active agent of 6.7 μg/cm²/hr or greater.

23. The formulation according to claim 1, further comprising a release liner.

24. A method for administering an antidementia active agent to a subject, said method comprising:
(a) applying to a skin site of said subject a transdermal antidementia active agent formulation, said formulation comprising:
(i) a backing;
(ii) a polymeric active agent reservoir layer comprising an antidementia active agent, wherein said antidementia active agent is present as both a freebase and as a salt; and
(iii) an adhesive layer comprising said antidementia active agent as freebase; and
(b) maintaining said formulation at said skin site of said subject for a period of time sufficient to deliver said active agent to said subject.

25. The method according to claim 24, wherein said antidementia active agent is donepezil.

26-46. (canceled)

47. A kit comprising a transdermal antidementia active agent formulation, said formulation comprising:
a backing;
an active agent reservoir layer comprising an antidementia active agent, wherein said antidementia active agent is present as a freebase; and
an adhesive layer comprising said antidementia active agent.

48. The kit according to claim 47, wherein said antidementia active agent is donepezil.

49-69. (canceled)
